(19) United States
(12) Patent Application Publication
(10) Pub. No.: US 2011/0230719 A1
(43) Pub. Date: Sep. 22, 2011

(54) IMAGE CAPTURING METHOD AND APPARATUS

(75) Inventors: Kazuhiko Katakura, Kanagawa-ken (JP); Hitoshi Shimizu, Kanagawa-ken (JP); Shinichi Shimotsu, Kanagawa-ken (JP)

(73) Assignee: FUJIFILM Corporation, Tokyo (JP)

(21) Appl. No.: 12/986,007

(22) Filed: Jan. 6, 2011

(30) Foreign Application Priority Data
Mar. 19, 2010 (JP) 063577/2010

(51) Int. Cl. A61B 1/12

(52) U.S. Cl. 600/156

(57) ABSTRACT

In an image capturing apparatus in which irradiation light emitted from a light source is projected onto an observation area through an irradiation window, reflection light of the irradiation light incident on the irradiation window and reflected from the window is detected and a liquid is discharged onto the irradiation window according to a result of the detection.

### BACKGROUND OF THE INVENTION

1. Field of the Invention

The present invention relates to an image capturing method and apparatus for capturing an image by projecting irradiation light emitted from a light source onto an observation area through an irradiation window.

2. Description of the Related Art

When performing a surgical operation, it is necessary to pay attention not to damage a blood vessel, but a risk of damaging a blood vessel (bleeding risk) is present because a blood vessel located below a certain depth can not be recognized. Endoscopic surgery, in particular, has a higher bleeding risk than ordinary surgery because of a narrow surgical field that makes it difficult to understand the positional relationship between a tip of a treatment tool, such as an electrosurgical knife, an ultrasonic surgical knife, or the like, and a blood vessel, and has a problem that, if the bleeding is excessive, it is difficult to treat the bleeding, although it has the advantage that it is minimally invasive surgery. Therefore, the endoscopic surgery needs to be performed by a doctor skilled in endoscope operation.

Consequently, in order to reduce bleeding risks from blood vessels, a technology for obtaining a fluorescence image of blood vessels by administering a labeling reagent, such as an indocyanine green (ICG), into a living body and projecting excitation light having a specific wavelength for exiting the labeling reagent is known as described, for example, in PCT Japanese Publication No. 2003-510121.

Here, in endoscopic surgery, there may be a case in which a stain, such as blood or mucous membrane, adheres to a distal end portion of the endoscope due to bleeding during the surgical operation, and a stained tip portion of the endoscope causes a problem of poor visibility.

Further, in the method that illuminates a near infrared light as the excitation light, as described in PCT Japanese Publication No. 2003-510121, if a stain, such as blood or the like, adheres to an irradiation window provided on the tip of the endoscope, the stain is burnt to the window, causing a problem that required illumination can not be provided because the near infrared light has an extremely high power density in comparison with ordinary illumination light.

Japanese Unexamined Patent Publication No. 5 (1993)-199979 proposes an endoscope in which the insertion section is covered with a sheath having a gas/liquid feed tube to feed a liquid and a gas to an observation window provided on the tip of the endoscope, thereby allowing the observation window to be cleaned and dried during a surgical operation.

The method described in Japanese Unexamined Patent Publication No. 5 (1993)-199979 allows the operator to determine whether or not the tip of the insertion section is stained by observing an actually captured image. But, for example, where only a fluorescence image is obtained using near infrared light, as in the method described in PCT Japanese Publication No. 2003-510121, it may sometimes be difficult to instantaneously determine that the tip of the insertion section is stained.

Further, the use of near infrared light causes the problem of burned stain described above. It is, therefore, necessary to detect and remove a stain immediately, but such operation is difficult for the method described in Japanese Unexamined Patent Publication No. 5 (1993)-199979.

Japanese Unexamined Patent Publication No. 2001-046324 discloses a method for detecting a stain on the tip of the scope by providing a light guide, different from the illumination light guide, on the tip of the scope and detecting a stain, such as blood or mucous membrane, adhered to the light guide.

The method described in Japanese Unexamined Patent Publication No. 2001-046324, however, provides only a warning display when a stain on the tip of the scope is detected, and the operator who saw the warning display needs to withdraw the scope from the body cavity and wipe out the stain.

That is, Japanese Unexamined Patent Publication No. 2001-046324 does not envisage the burned stain describe above at all, and a stain can not be removed immediately by the withdrawal of the scope by the operator after the warning display, so that the problem of burned stain can not be avoided.

Further, Japanese Unexamined Patent Publication No. 2001-046324 detects a stain by a light guide different from the illumination light guide so that a stain adhered to the illumination light guide can not be detected if a stain does not adhere to the stain detection light guide.

The present invention has been developed in view of the circumstances described above, and it is an object of the present invention to provide an image capturing method and apparatus capable of effectively preventing a burned stain by instantaneously detecting and removing a stain from an irradiation window even when illumination light having a high power density is used.

### SUMMARY OF THE INVENTION

An image capturing method of the present invention is a method, including the steps of:

projecting irradiation light emitted from a light source onto an observation area through an irradiation window;

capturing an image by receiving light from the observation area irradiated with the irradiation light;

detecting reflection light of the irradiation light incident on the irradiation window and reflected from the window; and

discharging a liquid onto the irradiation window according to a result of the detection.

An image capturing apparatus of the present invention is an apparatus including:

a light projection unit for projecting irradiation light emitted from a light source onto an observation area through an irradiation window;

an imaging unit for capturing an image by receiving light from the observation area irradiated with the irradiation light;

a reflection light detection unit for detecting reflection light of the irradiation light incident on the irradiation window and reflected from the irradiation window; and

an irradiation window cleaning unit for discharging a liquid onto the irradiation window according to a result of the detection by the reflection light detection unit.

In the image capturing apparatus of the present invention described above, the light projection unit may include a body cavity insertion unit to be inserted into a body cavity, and the irradiation window may be provided on a tip of the body cavity insertion unit.

Further, the light projection unit may be a unit that projects excitation light in an invisible wavelength range and aiming light in a visible wavelength range for indicating an irradiation position of the excitation light, as the irradiation light, to the observation area through respective irradiation windows, the imaging unit may be a unit that captures a fluorescence image by receiving fluorescence emitted from the observation area irradiated with the excitation light, and the reflection light detection unit may be a unit that detects reflection light of the aiming light.

Still further, a common irradiation window may be used as the irradiation window through which the excitation light is projected and the irradiation window through which the aiming light is projected.

Further, the light projection unit may be a unit that projects excitation light, as the irradiation light, to the observation area through the irradiation window, the imaging unit may be a unit that captures a fluorescence image by receiving fluorescence emitted from the observation area irradiated with the excitation light, and the reflection light detection unit may be a unit that detects reflection light of the excitation light.

Still further, the light projection unit may be a unit that projects white light, as the irradiation light, to the observation area through the irradiation window, the imaging unit may be a unit that captures an ordinary image by receiving reflection light reflected from the observation area irradiated with the white light, and the reflection light detection unit may be a unit that detects reflection light of the white light.

Further, the irradiation window cleaning unit may be a unit that discharges a gas onto the irradiation window.

According to the image capturing method and apparatus of the present invention, reflection light of irradiation light incident on an irradiation window and reflected from the irradiation window is detected, and a liquid is discharged onto the irradiation window according to a result of the detection. This allows a burned stain to be effectively prevented by instantaneously detecting and removing a stain on the irradiation window even when high power density irradiation light is used.

Further, in the image capturing apparatus of the present invention, if excitation light in an invisible wavelength range and aiming light in a visible wavelength range for indicating an irradiation position of the excitation light are projected, as the irradiation light, to the observation area through respective irradiation windows, and a liquid is discharged onto the irradiation window according to a result of the detection of reflection light of the aiming light, stain detection may be performed in synchronization with the projection of the excitation light. Where reflection light of the excitation light reflected from the irradiation window is monitored directly, for example, in a case in which excitation light is inputted to the irradiation window and reflection light of the excitation light is guided to a detector using an optical fiber coupler, excitation light power loss will occur in the optical fiber coupler which necessitates an additional excitation light source, resulting in increased cost. But the scheme of detecting reflection light of the aiming light described above may prevent such problems.

Still further, when a common irradiation window is used as the irradiation window through which the excitation light is projected and the irradiation window through which the aiming light is projected, a stain on the irradiation window for the excitation light may be detected appropriately.

Further, if the excitation light is projected onto the observation area through the irradiation window and a liquid is discharged onto the irradiation window according to a result of the detection of reflection light of the excitation light, reflection light of high power density excitation light can be monitored directly, so that a burned stain may be prevented more effectively.

Still further, if white light is projected onto the observation area through the irradiation window and a liquid is discharged onto the irradiation window according to a result of the detection of reflection light of the white light, stain detection may be made appropriately while observing an ordinary image.

### BRIEF DESCRIPTION OF THE DRAWINGS

FIG. 1 is a schematic configuration diagram of a rigid endoscope system that employs a first embodiment of the image capturing apparatus of the present invention.

FIG. 2 is a schematic configuration diagram of the body cavity insertion unit shown in FIG. 1.

FIG. 3 is a schematic configuration diagram of a tip portion of the body cavity insertion unit.

FIG. 4 is a schematic configuration diagram of a tip portion of the body cavity insertion unit.

FIG. 5 is a cross-sectional view taken along the line 5-5′ in FIG. 3.

FIG. 6 illustrates a spectrum of light outputted from each light projection unit of the body cavity insertion unit and spectra of fluorescence and reflection light emitted/reflected from an observation area irradiated with the light.

FIG. 7 is a schematic configuration diagram of an imaging unit.

FIG. 8 illustrates spectral sensitivity of the imaging unit.

FIG. 9 is a block diagram of a processor and a light source unit, illustrating schematic configurations thereof.

FIG. 10 is a schematic configuration diagram of a gas/liquid feed unit.

FIG. 11 is a flowchart for explaining an operation of the rigid endoscope system according to the first embodiment of the present invention.

FIG. 12 is a block diagram of an image processing unit and a light source unit of a rigid endoscope system that employs a second embodiment of the image capturing apparatus of the present invention.

FIG. 13 is a block diagram of an image processing unit and a light source unit of a rigid endoscope system that employs a third embodiment of the image capturing apparatus of the present invention.

### DESCRIPTION OF THE PREFERRED EMBODIMENTS

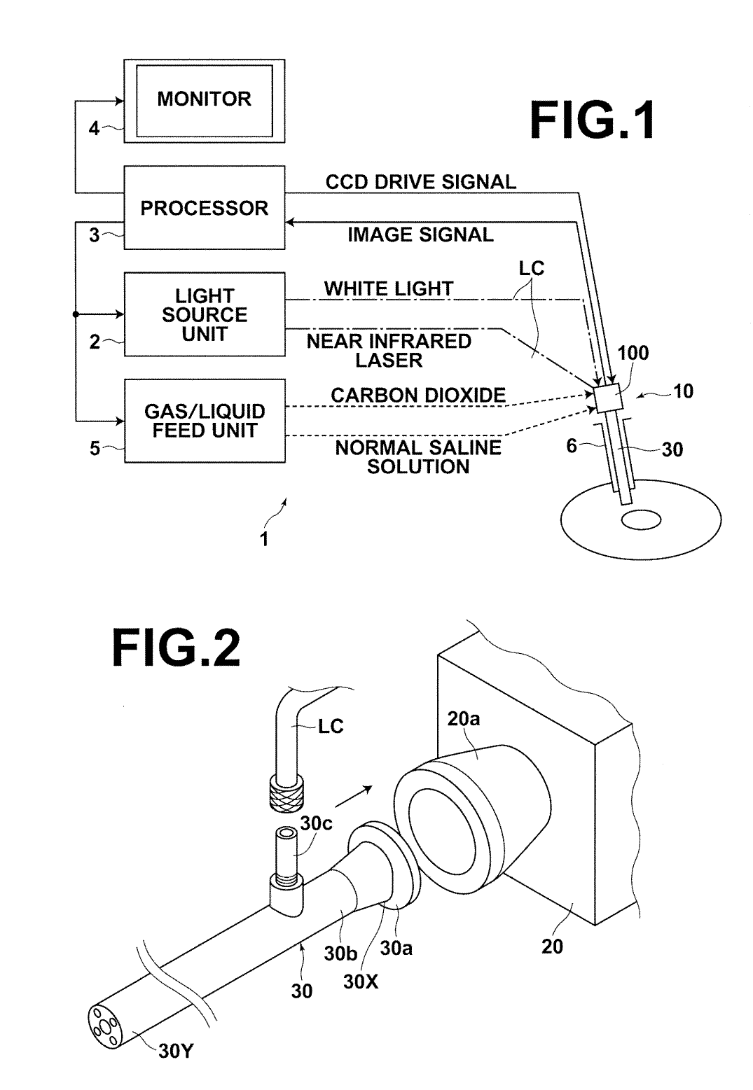

Hereinafter, a rigid endoscope system that employs a first embodiment of the image capturing apparatus of the present invention will be described with reference to the accompanying drawings. The present invention specifically relates to a structure for detecting and cleaning a stain on an insertion section to be inserted into a body, but the structure of the overall system will be described first. FIG. 1 is an overview of rigid endoscope system 1 of the present embodiment, illustrating a schematic configuration thereof.

As shown in FIG. 1, rigid endoscope system 1 of the present embodiment includes light source unit 2 for emitting blue light and near infrared light, rigid endoscope imaging device 10 for projecting white light obtained through wavelength conversion of the blue light, and near infrared light emitted from light source unit 2 onto an observation area and capturing an ordinary image based on reflection light reflected from the observation area irradiated with the white light and a fluorescence image based on fluorescence emitted from the observation area irradiated with the near infrared light, processor 3 for performing predetermined processing on image signals obtained by rigid endoscope imaging device 10, monitor 4 for displaying a fluorescence image and an ordinary image of the observation area based on a display control signal generated in processor 3, and gas/liquid feed unit 5 for feeding normal saline and carbon dioxide to rigid endoscope imaging device 10.

As shown in FIG. 1, rigid endoscope imaging device 10 includes body cavity insertion unit 30 to be inserted into an abdominal cavity or a chest cavity and imaging unit 20 for capturing an ordinary image and a florescence image of an observation area guided by the body cavity insertion unit 30.

Body cavity insertion unit 30 and imaging unit 20 are detachably connected, as shown in FIG. 2. Body cavity insertion unit 30 includes connection member 30a, insertion member 30b, and cable connection port 30c.

Connection member 30a is provided at first end 30X of body cavity insertion unit 30 (insertion member 30b), and imaging unit 20 and body cavity insertion unit 30 are detachably connected by fitting connection member 30a into, for example, aperture 20a formed in imaging unit 20.

Insertion member 30b is a member to be inserted into a body cavity when imaging is performed in the body cavity. Insertion member 30b is formed of a rigid material and has, for example, a cylindrical shape with a diameter of about 5 mm. Insertion member 30b accommodates inside thereof a group of lenses for forming an image of an observation area, and an ordinary image and a fluorescence image of the observation area inputted from second end 30Y are inputted, through the group of lenses, to imaging unit 20 on the side of first end 30X.

Cable connection port 30c is provided on the side surface of insertion member 30b and an optical cable LC is mechanically connected to the port. This causes light source unit 2 and insertion member 30b to be optically coupled through the optical cable LC.

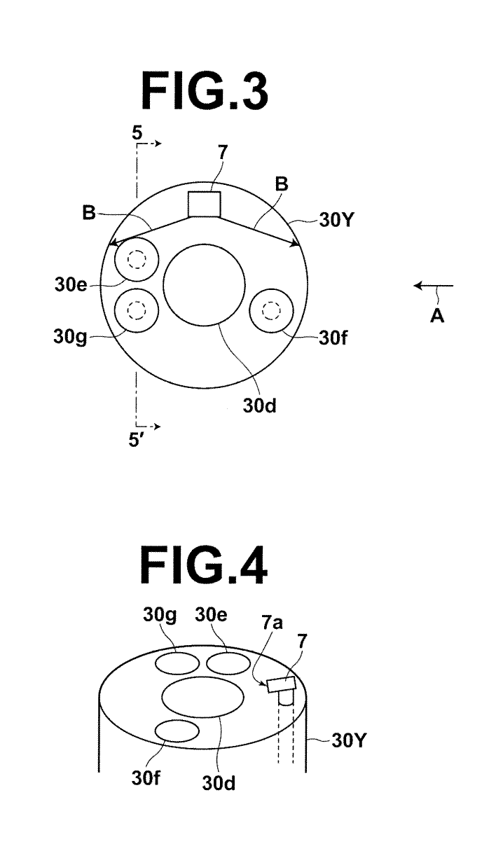

FIGS. 3 and 4 illustrate the second end 30Y of body cavity insertion unit 30. FIG. 4 is a perspective view of body cavity insertion unit 30 shown in FIG. 3 viewed from the arrow A.

As illustrated in FIGS. 3 and 4, imaging lens 30d is provided in the approximate center of second end 30Y of body cavity insertion unit 30 for forming an ordinary image and a fluorescence image, and white light irradiation lenses 30e and 30f for outputting white light are provide substantially symmetrically across the imaging lens 30d. The reason why two white light irradiation lenses are provide symmetrically with respect to imaging lens 30d is to prevent a shadow from being formed in an ordinary image due to irregularity of the observation area.

Further, near infrared light irradiation lens 30g for outputting near infrared light and aiming light for indicating the irradiation point of the invisible near infrared light is provided on second end 30Y of body cavity insertion unit 30.

Still further, second end 30Y of body cavity insertion unit 30 includes cleaning nozzle 7 for jetting normal saline or carbon dioxide supplied from gas/liquid feed unit 5.

In the present embodiment, the gas/liquid feed pipe is mounted inside of the insertion section of the body cavity insertion unit 30. But, a configuration may be adopted in which the gas/liquid feed pipe is provided in a sheath covering body cavity insertion unit 30 and one end of which is connected to the cleaning nozzle while the other end of which is connected to gas/liquid feed unit 5, thereby letting the normal saline or carbon dioxide supplied from gas/liquid feed unit 5 onto the imaging lens and irradiation lens from the nozzle opening.

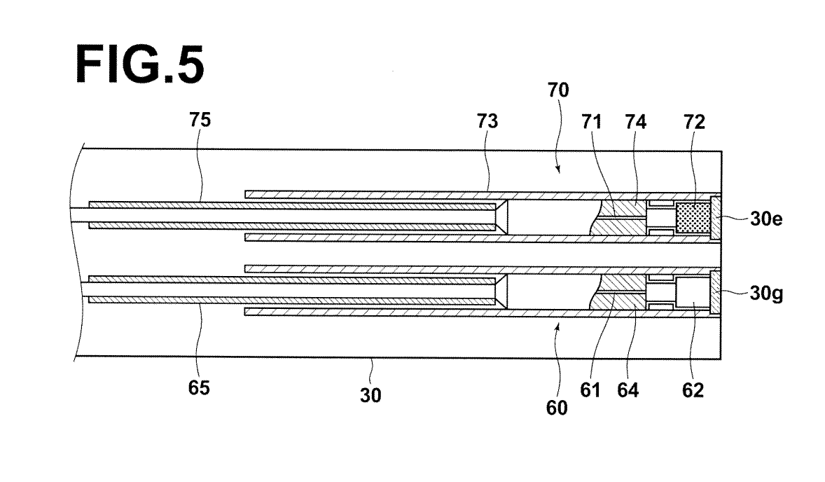

FIG. 5 is a cross-sectional view taken along the line 5-5′ in FIG. 3. As illustrated in FIG. 5, body cavity insertion unit 30 includes inside thereof white light projection unit 70 and near infrared light projection unit 60. White light projection unit 70 includes multimode optical fiber 71 for guiding the blue light and fluorescent body 72 which is excited by absorbing a portion of the blue light guided through multimode optical fiber 71 and emits visible light of green to yellow. Fluorescent body 72 is formed of a plurality of types of fluorescent materials, such as a YAG fluorescent material, BAM (BaMgAl₁₀O₁₇), and the like.

Tubular sleeve member 73 is provided so as to cover the periphery of fluorescent body 72, and ferrule 74 for holding multimode optical fiber 71 as the central axis is inserted in sleeve member 73. Further, flexible sleeve 75 is inserted between sleeve member 73 and multimode optical fiber 71 extending from the proximal side (opposite to the distal side) of ferrule 74 to cover the jacket of the fiber.

Near infrared light projection unit 60 includes multimode optical fiber 61 for guiding the near infrared light and space 62 is provided between multimode optical fiber 61 and near infrared light irradiation lens 30g.

Also blue light projection unit 60 is provided with tubular sleeve member 63 covering the periphery of space 62, in addition to ferrule 64 and flexible sleeve 65, as in white light projection unit 70.

The dotted circle in each irradiation lens shown in FIG. 3 represents the output end of the multimode optical fiber. As for the multimode optical fiber used in each light projection unit, for example, a thin optical fiber with a core diameter of 105 μm, a clad diameter of 125 μm, and an overall diameter, including a protective outer jacket, of 0.3 mm to 0.5 mm may be used.

Each spectrum of light projected to an observation area from each light projection unit, and spectra of fluorescence and reflection light emitted/reflected from the observation area irradiated with the light are shown in FIG. 6. FIG. 6 shows a blue light spectrum S1 outputted through fluorescent body 72 of white light projection unit 70, a green to yellow visible light spectrum S2 excited and emitted from fluorescent body 72 of white light projection unit 70, a near infrared light spectrum 53 outputted from a first or second near infrared light projection unit, and an ICG fluorescence spectrum S4 emitted from an observation area irradiated with the near infrared light spectrum 53 outputted from the first or second near infrared light projection unit.

The term “white light” as used herein is not strictly limited to light having all wavelength components of visible light and may include any light as long as it includes light in a specific wavelength range, for example, primary light of R (red), G (green), or B (blue). Thus, in a broad sense, the white light may include, for example, light having wavelength components from green to red, light having wavelength components from blue to green, and the like. Although white light projection unit 70 outputs the blue light spectrum S1 and visible light spectrum S2 shown in FIG. 6, the light of these spectra is also regarded as white light.

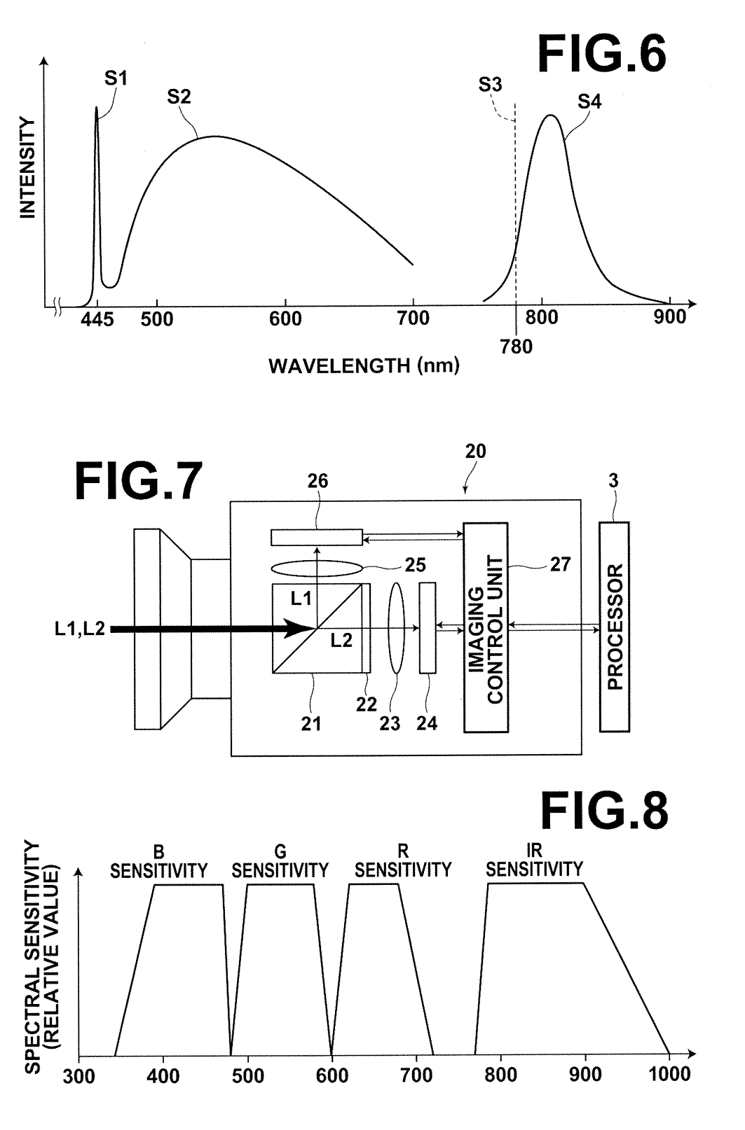

FIG. 7 shows a schematic configuration of imaging unit 20. Imaging unit 20 includes a first imaging system for generating a fluorescence image signal by capturing a fluorescence image of an observation area formed by the group of lenses inside of body cavity insertion unit 30 and a second imaging system for generating an ordinary image signal by capturing an ordinary image of the observation area formed by the group of lenses inside of body cavity insertion unit 30. These imaging systems are separated into two orthogonal optical axes by dichroic prism 21 having a spectroscopic property that reflects the ordinary image and transmits the fluorescence image.

The first imaging system includes excitation light cut filter 22 that transmits an fluorescence image outputted from body cavity insertion unit 30 and cuts the excitation light, first image forming system 23 that forms fluorescence image L2 outputted from body cavity insertion unit 30 and transmitted through dichroic mirror 21 and excitation light cut filter, and high sensitivity image sensor 24 that captures fluorescence image L2 formed by first image forming system 23.

The second imaging system includes second image forming system that forms ordinary image L1 outputted from body cavity insertion unit 30 and reflected from dichroic mirror 21 and image sensor 26 that captures ordinary image L1 formed by second image forming system 25.

High sensitivity image sensor 24 is a sensor having a high sensitivity for detecting light in the wavelength range of fluorescence image L2 and outputting the detected light after converting the light to a fluorescence image signal.

Ordinary image sensor 26 is a sensor for detecting light in the wavelength range of ordinary image L2 and outputting the detected light after converting the light to an ordinary image signal. Color filters of three primary colors, red (R), green (G), and blue (B) are arranged on the imaging surface of ordinary image sensor 26 in a Beyer or honeycomb pattern.

Referring now to FIG. 8, there is provided a graph of spectral sensitivity of imaging unit 20. More specifically, imaging unit 20 is configured such that the first imaging system has IR (near infrared) sensitivity while the second imaging system has R (red) sensitivity, G (green) sensitivity, and B (blue) sensitivity.

Imaging unit 20 further includes imaging control unit 27. Imaging control unit 27 is a unit that drive controls high sensitivity image sensor 24 and image sensor 26 based on a CCD drive control signal outputted from processor 3 and performs CDS/AGC (correlated double sampling/automatic gain control) and A/D conversion on a fluorescence image signal outputted from high sensitivity image sensor 24 and an ordinary image signal outputted from image sensor 26, and outputs the resultant image signals to processor 3 through a cable.

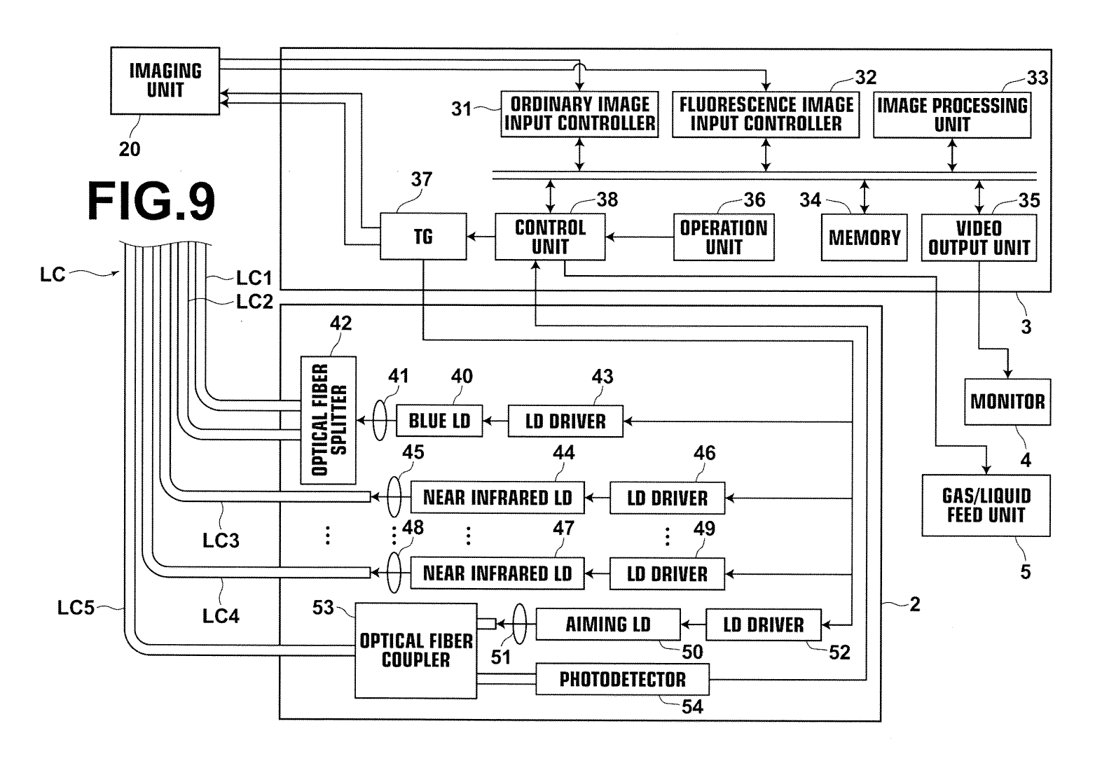

FIG. 9 is a block diagram of light source unit 2 and processor 3. As shown in FIG. 9, processor 3 includes ordinary image input controller 31, fluorescence image input controller 32, image processing unit 33, memory 34, video output unit 35, operation unit 36, TG (timing generator) 37, and control unit 38.

Ordinary image input controller 31 and fluorescence image input controller 32 are each provided with a line buffer having a predetermined capacity and temporarily store ordinary image signals and fluorescence image signals respectively outputted from imaging control unit 27 of imaging unit 20 with respect to each frame. Then, the ordinary image signals stored in ordinary image input controller 31 and the fluorescence image signals stored in fluorescence image input controller 32 are stored in memory 34 via the bus.

Image processing unit 33 receives the ordinary image signals and fluorescence image signals with respect to each frame read out from memory 34, then performs predetermined processing on these image signals, and outputs the resultant image signals to the bus.

Video output unit 35 receives the ordinary image signals and fluorescence image signals outputted from image processing unit 33 via the bus, generates display control signals by performing predetermine processing on the received signals, and outputs the display control signals to monitor 4.

Operation unit 36 receives various types of operation instructions, such as a mode switching instruction between ordinary image capturing mode and fluorescence image capturing mode and the like, and control parameters from the operator. TG 37 outputs drive pulse signals for driving high sensitivity image sensors 24 and image sensor 26 of imaging unit 20, and LD drivers 43, 46, 49, and 52 of light source unit 2, to be described later.

Control unit 38 performs overall control of the system. In addition, it outputs a control signal to gas/liquid feed unit 5 for air or water supply based on a detection signal of the reflection light detected by photodetector 54, to be described later, provided inside of light source unit 2.

As shown in FIG. 9, light source unit 2 includes blue LD light source 40 that emits 445 nm blue light, condenser lens 41 that condenses and inputs the blue light emitted from blue LD light source 40 to optical fiber splitter 42, optical fiber splitter 42 that simultaneously inputs the blue light inputted by condenser lens 41 to optical cable LC1 and optical cable LC2, and LD driver 43 that drives blue LD light source 40.

Optical cables LC1 and LC2 are optically coupled to multimode optical fibers 71 of white light projection unit 70 respectively.

Light source unit 2 further includes a plurality of near infrared LD light sources 44, 47 that emits 750 to 790 nm near infrared light, a plurality of condenser lenses 45, 48, each of which condenses and inputs near infrared light outputted from each of near infrared LD light sources 44, 47 to each of optical cables LC3, LC4, and a plurality of LD drivers 46, 49 that drives near infrared LD light sources 44, 47 respectively.

Although FIG. 9 illustrates only two sets of near infrared LD light source, condenser lens, and LD driver, but it is assumed that seven sets of these are provided in the present embodiment. Further, it is assumed that near infrared light condensed by the condenser lens of each set is inputted to each corresponding optical cable, and seven optical cables are bundled and optically coupled to multimode optical fiber 61 of near infrared light projection unit 60 in body cavity insertion unit 30.

In the present embodiment, near infrared light is used as the excitation light, but it is not limited to this and is determined appropriately according to the type of fluorochrome administered to the subject or the type of living tissue for causing autofluorescence.

Light source unit 2 further includes aiming LD light source 50 that emits about 635 nm aiming light, condenser lens 51 that condenses and inputs the aiming light outputted from aiming LD light source 50 to optical fiber coupler 53, optical fiber coupler 53 that inputs the aiming light inputted by condenser lens 51 to optical cable LC5 and inputs reflection light of the aiming light reflected from near infrared light irradiation lens 30g and inputted through optical cable LC5 to photodetector 54, LD driver 52 that drives aiming LD light source 50, and photodetector 54 described above.

The wavelength of aiming LD light source 50 is determined to 635 nm in the present embodiment from the viewpoint of availability of the parts, but any wavelength in the visible wavelength range from 400 nm to 700 nm may be used. Further, a LID is used as the aiming light source in view of the coupling efficiency of the optical fiber, but a LED (light emitting diode) or lamp source that emits white light may also be used.

Optical cable LC5 is bundled together with optical cables LC3, CL4 and optically coupled to multimode optical fiber 61 of near infrared projection unit 60.

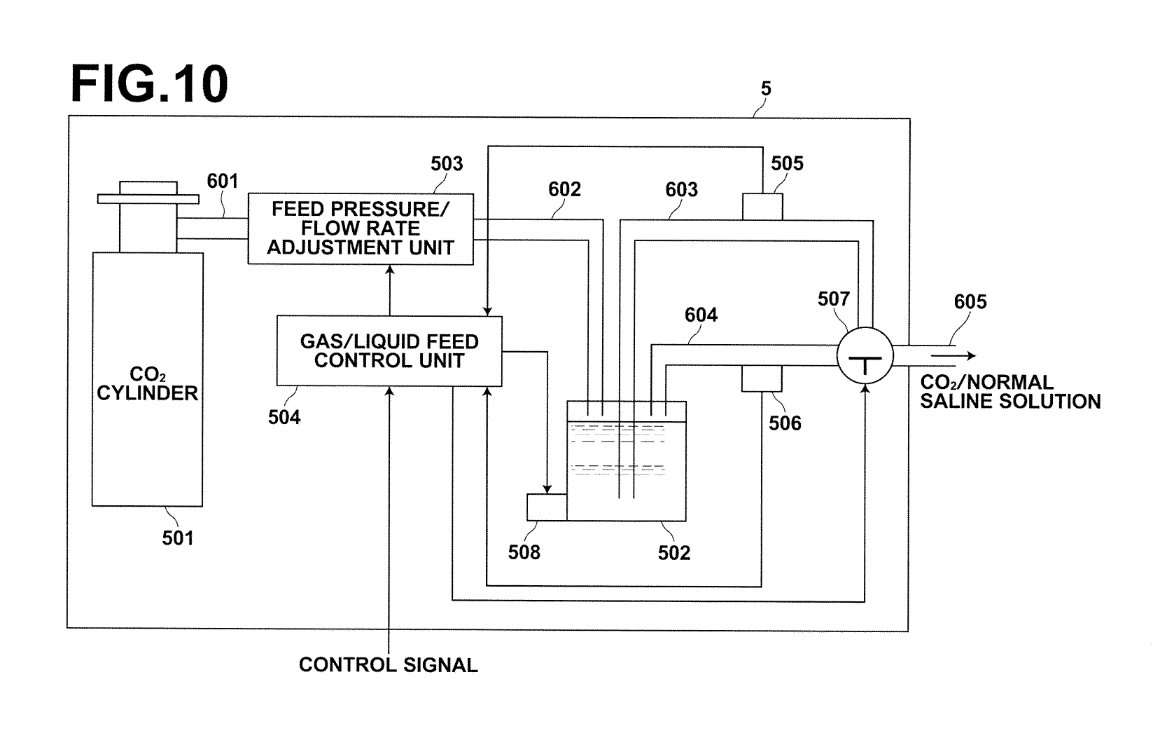

FIG. 10 is a schematic configuration diagram of gas/liquid feed unit 5. As shown in FIG. 10, gas/liquid feed unit 5 includes CO₂ cylinder 501, liquid storage tank 502, feed pressure/flow rate adjustment unit 503, gas/liquid feed control unit 504, flow sensors 505, 506, three-way valve 507, and heater 508.

CO₂ cylinder 501 stores CO₂ gas which is sent to feed pressure/flow rate adjustment unit 503 through pipe 601. Feed pressure/flow rate adjustment unit 503 feeds the CO₂ gas to liquid storage tank 502 through pipe 602 after regulating the pressure and flow rate thereof. Liquid storage tank 502 stores, for example, a normal saline solution, and is structured to allow the normal saline solution to be sent to pipe 603 by the CO₂ gas through pipe 602. Further, liquid storage tank 502 is capable of sending the CO₂ gas to pipe 604.

Three-way valve 507 sends the normal saline solution from pipe 603 or CO₂ gas from pipe 604 to gas/liquid feed pipe 605 by switching them, thereby feeding the normal saline solution or CO₂ gas to cleaning nozzle 7 through gas/liquid feed pipe 605.

Flow sensor 505 is a sensor for detecting the flow rate of the normal saline solution flowing through pipe 603 while flow sensor 506 is a sensor for detecting the flow rate of the CO₂ gas flowing through pipe 604.

Heater 508 is a heater for maintaining the normal saline solution in the liquid storage tank 502 at a predetermined temperature and has a built-in temperature sensor.

Gas/liquid feed control unit 504 controls feed pressure/flow rate adjustment unit 503, three-way valve 507, and heater 508 based on a control signal outputted from control unit 38 of processor 3 and detection signals from flow sensors 505 and 506.

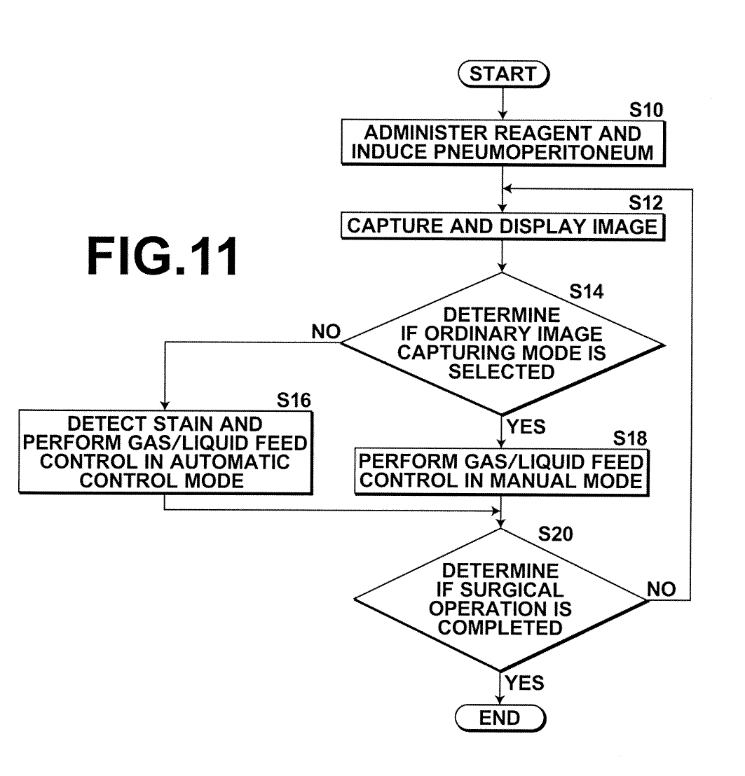

An operation of the rigid endoscope system of the present embodiment will now be described with reference to the flowchart of FIG. 11.

First, a labeling reagent is administered to a subject from the vein and a CO₂ gas is fed into a body cavity by gas/liquid feed unit 5 to induce pneumoperitoneum. Here, gas/liquid feed control unit 504 controls heater 508 to maintain the normal saline solution at a predetermined temperature (S10).

Then, at least one of a fluorescence image capturing mode and an ordinary image capturing mode is selected by the operator using operation unit 36 of processor 3, and the selection signal is received by control unit 38, whereby each system component is controlled by the control unit 38 and a florescence image and/or an ordinary image is captured and displayed (S12). Note that, in the present embodiment, both the fluorescence image capturing mode and ordinary image capturing mode can be selected at the same time, as well as selecting either one of them.

Hereinafter, operations of the rigid endoscope system when only the ordinary image capturing mode is selected, when both the ordinary image capturing mode and fluorescence image capturing mode are selected, and when only the fluorescence image capturing mode is selected will be described.

First, an operation of the system when only the ordinary image capturing mode is selected will be described.

When only the ordinary image capturing mode is selected, blue light emitted from blue LD light source 40 of light source unit 2 is inputted to optical cables LC1, LC2 at the same time through condenser lens 41 and optical fiber splitter 42. Then, the blue light is guided through optical cables LC1, LC2, inputted to body cavity insertion unit 30, and guided through multimode optical fiber 71 of white light projection unit 70 in body cavity insertion unit 30. A portion of the blue light outputted from the output end of the multimode optical fiber 71 is transmitted through fluorescent body 72 and projected onto the observation area, while the remaining blue light other than the portion is subjected to wavelength conversion to green to yellow visible light by fluorescent body 72 and the visible light is projected onto the observation area. That is, the observation area is irradiated with white light formed of the blue light and green to yellow visible light.

An ordinary image reflected from the observation area irradiated with the white light is inputted from imaging lens 30d at the tip 30Y of insertion member 30b, then guided by the group of lenses inside of the insertion member 30b, and outputted to imaging unit 20.

The ordinary image inputted to imaging unit 20 is reflected in a right angle direction by dichroic prism 21 and formed on the imaging surface of image sensor 26 by second image forming system 25, and captured by image sensor 26.

Then, R, G, and B image signals outputted from image sensor 26 are subjected to CDS/AGC (correlated double sampling/automatic gain control) and A/D conversion in imaging control unit 27, and outputted to processor 3 through cable 5.

The ordinary image signals inputted to processor 3 are temporarily stored in ordinary image input controller 31 and then stored in memory 34. Then, ordinary image signals with respect to each frame read out from memory 34 are subjected to tone correction and sharpness correction in image processing unit 33 and sequentially outputted to video output unit 35.

Video output section 35 generates display control signals by performing predetermined processing on the inputted ordinary image signals and outputs the display control signals with respect to each frame to monitor 4. Monitor 4, in turn, displays an ordinary image based on the inputted display control signals.

Next, an operation of the system when both the ordinary image capturing mode and fluorescence image capturing mode are selected will be described.

When both the ordinary image capturing mode and fluorescence image capturing mode are selected, fluorescence image capturing and displaying, to be described later, are performed simultaneously with the ordinary image capturing and displaying described above. In addition, aiming image capturing is also performed and the captured aiming image is displayed superimposed on the ordinary image.

More specifically, when capturing a fluorescence image, near infrared light emitted from near infrared LD light sources 44, 47 is inputted to optical cable LC3, LC4 through condenser lenses 45, 48. Then, the infrared light is guided through optical cables LC3, LC4, inputted to body cavity insertion unit 30, guided through multimode optical fiber 61 of near infrared light projection unit 60 in body cavity insertion unit 30, and projected onto an observation area.

An ICG fluorescence image emitted from the observation area irradiated with excitation light of the near infrared light is inputted from imaging lens 30d at the tip 30Y of insertion member 30b, then guided by the group of lenses inside of the insertion member 30b, and outputted to imaging unit 20.

The ICG fluorescence image inputted to imaging unit 20 is transmitted through dichroic prism 21 and excitation light cut filter 22 and formed on the imaging plane of high sensitivity image sensor 24 by first image forming system 23 and captured by high sensitivity image sensor 24. The ICG fluorescence image signal outputted from high sensitivity image sensor 24 is subjected to CDS/AGC (correlated double sampling/automatic gain control) and A/D conversion in imaging control unit 27, and outputted to processor 3 through cable 5.

The florescence image signals inputted to processor 3 are temporarily stored in fluorescence image input controller 32 and then stored in memory 34. Then, fluorescence image signals with respect to each frame read out from memory 34 are subjected to predetermined processing in image processing unit 33 and sequentially outputted to video output unit 35.

Video output section 35 generates display control signals by performing predetermined processing on the inputted florescence image signals and outputs the display control signals with respect to each frame to monitor 4. Monitor 4, in turn, displays a fluorescence image based on the inputted display control signals.

Here, an aiming image is further captured together with the capturing of the ordinary and fluorescence images described above.

More specifically, aiming light emitted from aiming LD light source 50 of light source unit 2 is inputted to optical cable LC5 through condenser lens 51 and optical fiber coupler 53, then inputted to body cavity insertion unit 30 through optical cable LC5, guided through multimode optical fiber 61 of near infrared light projection unit 60 in body cavity insertion unit 30, and projected onto the observation area.

Then, aiming image capturing is performed together with the ordinary image capturing described above. More specifically, an aiming image emitted from the observation area irradiated with the aiming light is inputted from imaging lens 30d at the tip 30Y of insertion member 30b, then guided by the group of lenses inside of the insertion member 30b, and outputted to imaging unit 20.

The aiming image inputted to imaging unit 20 is reflected in a right angle direction by dichroic prism 21 and formed on the imaging surface of image sensor 26 by second image forming system 25, and captured by image sensor 26.

Then, R, G, and B image signals outputted from image sensor 26 are subjected to CDS/AGC (correlated double sampling/automatic gain control) and A/D conversion in imaging control unit 27, and outputted to processor 3 through cable 5.

The aiming image signals and ordinary image signals inputted to processor 3 are temporarily stored in ordinary image input controller 31 and then stored in memory 34. Then, aiming image signals and ordinary image signals with respect to each frame read out from memory 34 are subjected to tone correction and sharpness correction in image processing unit 33 and sequentially outputted to video output unit 35.

Video output section 35 generates display control signals by performing predetermined processing on the inputted aiming image signals and ordinary image signals and outputs the display control signals with respect to each frame to monitor 4. Monitor 4, in turn, displays a composite image formed of an aiming image and an ordinary image superimposed on top of each other based on the inputted display control signals.

Next, an operation of the system when only the fluorescence image capturing mode is selected will be described.

When only the fluorescence image capturing mode is selected, the fluorescence image capturing and displaying described above are performed and aiming light is projected onto the observation area. Note that, here, an aiming image is not captured although the aiming light is projected onto the observation area. The reason why the aiming light is projected to the observation area without capturing an aiming image is for detecting a stain on the tip of body cavity insertion unit 30 to be described later. The operation of the stain detection will be described later in detail.

As described above, an ordinary image, a fluorescence image, or a composite image of ordinary image and aiming image is captured and displayed according each imaging mode. Here, if a stain adheres to the tip of body cavity insertion unit 30, stain detection and removal is performed according to each imaging mode.

More specifically, when only the ordinary image capturing mode is selected or when both the ordinary image capturing mode and fluorescence image capturing mode are selected, that is, if ordinary image capturing mode is selected (S14, YES), control unit 38 of processor 3 causes a stain removal operation to be performed in manual mode (S18).

More specifically, when control unit 38 judges that the ordinary image capturing mode is selected, it outputs a control signal and, in response to the control signal, gas/liquid feed control unit 504 sets the gas/liquid feed control mode to manual mode.

Then, for example, when a judgment is made by the operator that a stain is attached to the ordinary image and a gas/liquid feed switch provided in operation unit 36 of processor 3 is operated by the operator, gas/liquid feed control unit 504 controls feed pressure/flow rate adjustment unit 503 and three-way valve 507 to allow manual gas/liquid feeding.

More specifically, if instructed by the operator to feed a liquid from cleaning nozzle 7, the pressure and flow rate of the CO₂ gas in CO₂ cylinder 501 are adjusted by feed pressure/flow rate adjustment unit 503 and the CO₂ gas is fed to liquid storage tank 502 through pipe 602, whereby the normal saline solution in liquid storage tank 502 is fed to pipe 603.

Then, the normal saline solution flowing through pipe 603 is passed through three-way valve 507 and fed to gas/liquid feed pipe 605. The normal saline solution fed to gas/liquid feed pipe 605 is fed to cleaning nozzle 7 through the pipe and discharged from the nozzle.

If instructed by the operator to feed a gas from cleaning nozzle 7, three-way valve 507 is switched to pipe 604 and the CO₂ gas passed through liquid storage tank 502 is fed to pipe 604. The CO₂ gas fed to pipe 604 is passed through three-way valve 507 and fed to gas/liquid feed pipe 605. The CO₂ gas fed to gas/liquid feed pipe 605 is fed to cleaning nozzle 7 through the pipe and discharged from the nozzle.

In the mean time, if only the fluorescence image capturing mode is selected (S14, NO), control unit 38 of processor 3 causes a stain detection operation and a stain removal operation to be performed in automatic mode (S16).

More specifically, when control unit 38 judges that only the fluorescence image capturing mode is selected, it starts monitoring a detection signal of reflection light detected by photodetector 54 in light source unit 2 and outputs a control signal to gas/liquid feed control unit 504, and in response to the control signal, gas/liquid feed control unit 504 sets the gas/liquid feed control mode to automatic mode.

Photodetector 54 in light source unit 2 detects reflection light of aiming light emitted from aiming LD light source 50 and inputted to near infrared light irradiation lens 30g at the tip of body cavity insertion unit 30 through optical cable LC5 reflected from near infrared light irradiation lens 30g due to a stain attached to the surface thereof and returned to optical cable LC5.

When the reflection light is detected by photodetector 54 and a detection signal thereof is received by control unit 38, a control signal is outputted from control unit 38 to gas/liquid feed control unit 504. Then, in response to the control signal, gas/liquid feed control unit 504 controls feed pressure/flow rate adjustment unit 503 and three-way valve 507 to cause automatic gas/liquid feeding. The amount of gas/liquid to be fed is predetermined, and gas/liquid feed control unit 504 outputs a control signal based on a signal from flow sensor 505 or flow sensor 506 so that the predetermined amount of gas/liquid is fed. A specific operation for the gas/liquid feeding is identical to that described above.

Then, the steps S12 to S18 are repeated until the surgical operation by rigid endoscope system 1 is completed (S20).

In the rigid endoscope system of the first embodiment described above, a gas/liquid is fed from cleaning nozzle 7 in manual mode when only the ordinary image capturing mode is selected or when both the ordinary image capturing mode and the fluorescence image capturing mode are selected, but the automatic control mode may be employed also in these cases.

More specifically, when only the ordinary image capturing mode is selected, aiming light is emitted from aiming LD light source 50, reflection light of the aiming light reflected from near infrared light irradiation lens 30g is detected by photodetector 54, and the gas/liquid feed control is performed automatically based on a detection signal of the reflection light detected by photodetector 54, as described above. When both the ordinary image capturing mode and fluorescence image capturing mode are selected, reflection light of the aiming light for capturing an aiming image reflected from near infrared light irradiation lens 30g is detected by photodetector 54, and the gas/liquid feed control may be performed automatically based on a detection signal of the reflection light detected by photodetector 54.

Next, a rigid endoscope system that employs a second embodiment of the image capturing apparatus of the present invention will be described. In the rigid endoscope system of the first embodiment, reflection light of aiming light is detected for detecting a stain on the tip of body cavity insertion unit 30, while in the rigid endoscope system of the second embodiment, reflection light of blue light, emitted from blue LD light source for capturing an ordinary image, reflected from white light irradiation lenses 30e, 30f is detected.

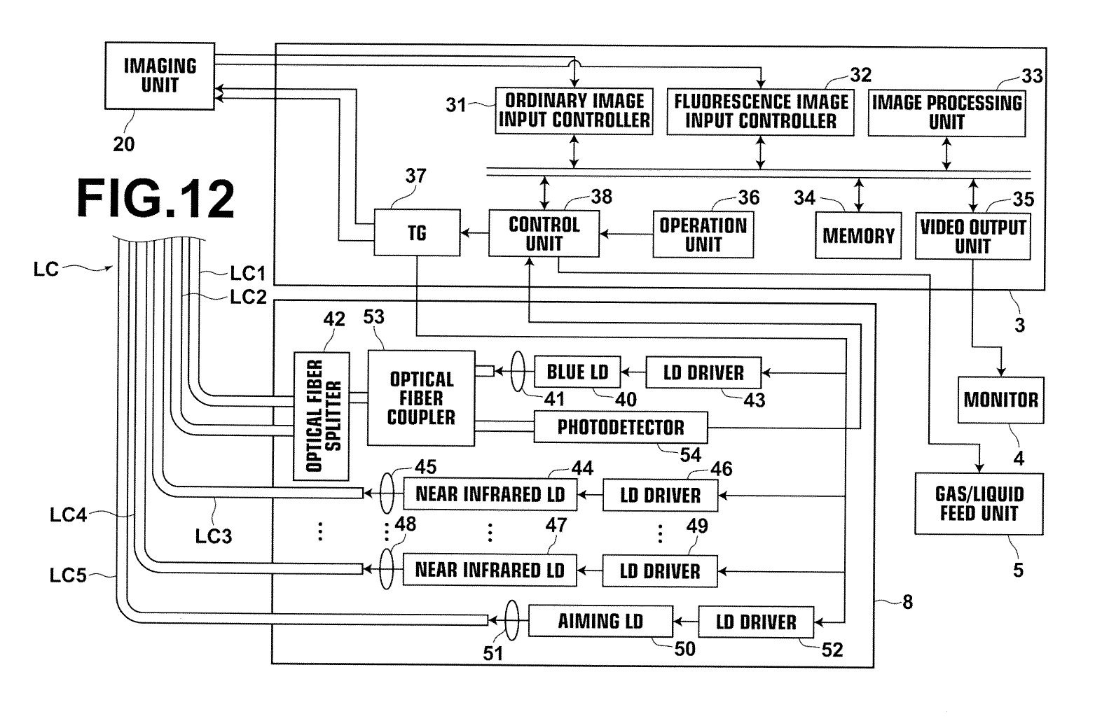

More specifically, in light source unit 8 of the rigid endoscope system of the second embodiment, optical fiber coupler 53 is disposed between blue LD light source 40 and optical fiber splitter 42, and photodetector 54 is optically coupled to optical fiber coupler 53, as shown in FIG. 12.

Reflection light of blue light emitted from blue LD light source 40 and inputted to white light irradiation lenses 30e, 30f at the tip of body cavity insertion unit 30 through optical fiber splitter 42 and optical cables LC1, LC2 reflected from white light irradiation lenses 30e, 30f due to a stain attached to the surface thereof and returned to optical cables LC1, LC2 is detected by photodetector 54.

Light source unit 8 has an identical structure to that of light source unit 2 of the first embodiment other than the positions of optical fiber coupler 53 and photodetector 54.

When the reflection light of the blue light is detected by photodetector 54 and a detection signal thereof is received by control unit 38, a control signal is outputted from control unit 38 to gas/liquid feed control unit 504 and gas/liquid feed control is performed by gas/liquid feed control unit 504.

In the rigid endoscope system of the second embodiment, even when only the fluorescence image capturing mode is selected, blue light is emitted from blue LD light source 40 to detect a stain on the tip of body cavity insertion unit 30 and gas/liquid feeding from cleaning nozzle 7 is performed automatically. In this case, however, ordinary image capturing is not performed.

In the rigid endoscope system of the second embodiment, a gas/liquid may be fed from nozzle 7 in manual mode, as in the first embodiment, when only the ordinary image capturing mode is selected or when both the ordinary image capturing mode and fluorescence image capturing mode are selected, or the automatic control mode may be employed also in these cases.

The other aspects of the rigid endoscope system of the second embodiment are identical to those of the rigid endoscope system of the first embodiment.

Next, a rigid endoscope system that employs a third embodiment of the image capturing apparatus of the present invention will be described. In the rigid endoscope system of the first embodiment, reflection light of aiming light is detected for detecting a stain on the tip of body cavity insertion unit 30, while in the rigid endoscope system of the third embodiment, reflection light of near infrared light, emitted from near infrared LD light sources for capturing fluorescence image, reflected from near infrared light irradiation lens 30g is detected.

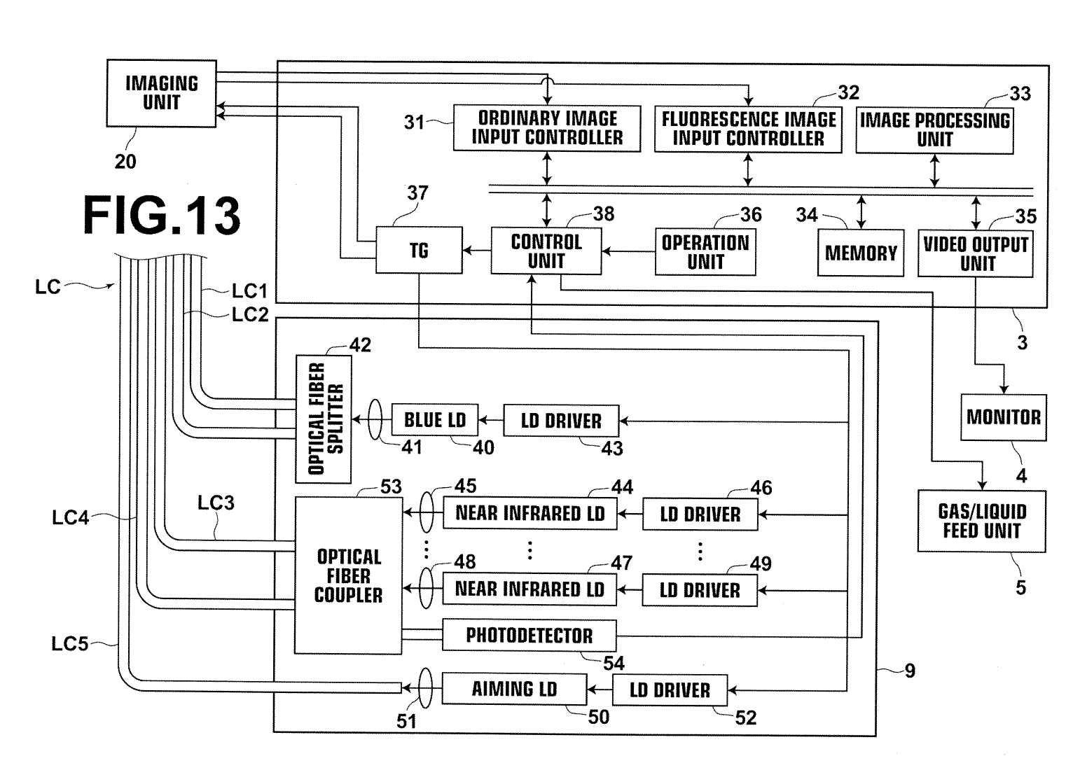

More specifically, in light source unit 9 of the rigid endoscope system of the third embodiment, optical fiber coupler 53 is disposed between near infrared LD light sources 44, 47 and optical cables LC3, LC4, and photodetector 54 is optically coupled to optical fiber coupler 53, as shown in FIG. 13.

Reflection light of near infrared light, emitted from near infrared LD light sources 44, 47 and inputted to near infrared light irradiation lenses 30g at the tip of body cavity insertion unit 30 through optical cables LC3, LC4, reflected from near infrared light irradiation lenses 30g due to a stain attached to the surface thereof and returned to optical cables LC3, LC4 is detected by photodetector 54.

Light source unit 9 has an identical structure to that of light source unit 2 of the first embodiment other than the positions of optical fiber coupler 53 and photodetector 54.

When the reflection light of the near infrared light is detected by photodetector 54 and a detection signal thereof is received by control unit 38, a control signal is outputted from control unit 38 to gas/liquid feed control unit 504 and gas/liquid feed control is performed by gas/liquid feed control unit 504.

In the rigid endoscope system of the third embodiment, gas/liquid feeding from cleaning nozzle 7 is set to automatic control mode when only the fluorescence image capturing mode is selected, as in the rigid endoscope system of the first embodiment. When only the ordinary image capturing mode is selected or when both the ordinary image capturing mode and fluorescence image capturing mode are selected, gas/liquid feeding from cleaning nozzle 7 may be performed in manual mode or in automatic control mode.

In the rigid endoscope system of the third embodiment, if feeding a gas/liquid from cleaning nozzle 7 in automatic control mode when only the ordinary image capturing mode is selected, near infrared light is emitted from near infrared LD light sources 44, 47 to detect a stain on the tip of body cavity insertion unit 30 even only the ordinary image capturing mode is selected and a gas/liquid is automatically fed from cleaning nozzle 7. In this case, however, fluorescence image capturing is not performed.

The other aspects of the rigid endoscope system of the third embodiment are identical to those of the rigid endoscope system of the first embodiment.

Further, in the embodiments described above, the image capturing apparatus of the present invention is applied to a rigid endoscope system, but the apparatus of the present invention may also be applied to other endoscope systems having a soft endoscope. Still further, the image capturing apparatus of the present invention is not limited to endoscope applications and may be applied to so-called video camera type medical image capturing systems without an insertion section to be inserted into a body cavity.

What is claimed is:

1. An image capturing method, comprising the steps of:
projecting irradiation light emitted from a light source onto an observation area through an irradiation window;
capturing an image by receiving light from the observation area irradiated with the irradiation light;
detecting reflection light of the irradiation light incident on the irradiation window and reflected from the window; and
discharging a liquid onto the irradiation window according to a result of the detection.

2. An image capturing apparatus, comprising:
a light projection unit for projecting irradiation light emitted from a light source onto an observation area through an irradiation window;
an imaging unit for capturing an image by receiving light from the observation area irradiated with the irradiation light;
a reflection light detection unit for detecting reflection light of the irradiation light incident on the irradiation window and reflected from the window; and
an irradiation window cleaning unit for discharging a liquid onto the irradiation window according to a result of the detection by the reflection light detection unit.

3. The image capturing apparatus of claim 2, wherein the light projection unit comprises a body cavity insertion unit to be inserted into a body cavity, and the irradiation window is provided on a tip of the body cavity insertion unit.

4. The image capturing apparatus of claim 2, wherein:
the light projection unit is a unit that projects excitation light in an invisible wavelength range and aiming light in a visible wavelength range for indicating an irradiation position of the excitation light, as the irradiation light, to the observation area through respective irradiation windows;
the imaging unit is a unit that captures a fluorescence image by receiving fluorescence emitted from the observation area irradiated with the excitation light; and
the reflection light detection unit is a unit that detects reflection light of the aiming light.

5. The image capturing apparatus of claim 4, wherein a common irradiation window is used as the irradiation window through which the excitation light is projected and the irradiation window through which the aiming light is projected.

6. The image capturing apparatus of claim 2, wherein:
the light projection unit is a unit that projects excitation light, as the irradiation light, to the observation area through the irradiation window;
the imaging unit is a unit that captures a fluorescence image by receiving fluorescence emitted from the observation area irradiated with the excitation light; and
the reflection light detection unit is a unit that detects reflection light of the excitation light.

7. The image capturing apparatus of claim 2, wherein:
the light projection unit is a unit that projects white light, as the irradiation light, to the observation area through the irradiation window;
the imaging unit is a unit that captures an ordinary image by receiving reflection light reflected from the observation area irradiated with the white light; and
the reflection light detection unit is a unit that detects reflection light of the white light.

8. The image capturing apparatus of claim 2, wherein the irradiation window cleaning unit is a unit that discharges a gas onto the irradiation window.
